(19) United States
(12) Patent Application Publication
(10) Pub. No.: US 2004/0256267 A1
(43) Pub. Date: Dec. 23, 2004

(54) Sterile screw delivery system

(76) Inventor: Gregory James Roger, New South Wales (AU)

Correspondence Address: JACOBSON HOLMAN PLLC, 400 SEVENTH STREET N.W., SUITE 600, WASHINGTON, DC, 20004, US

(21) Appl. No.: 10/488,863

(22) PCT Filed: Sep. 5, 2002
(86) PCT No.: PCT/AU02/01219

(30) Foreign Application Priority Data
Sep. 6, 2001 (AU) PR7541

(51) Int. Cl. A61B 17/06

(52) U.S. Cl. 206/438

(57) ABSTRACT

A holder (10) for a surgically implantable device such as a bone screw or nail. The holder (10) comprises a housing (11) having a top portion (12) and at least one side wall (13) depending from the top portion (12). The housing (10) further includes at least one internal receptacle member (14) having an elongate hollow body defining at a proximal end (16) an opening (17) in the top portion (12). The elongate hollow body extends to a distal end (18) spaced from the top portion (12).

### FIELD OF THE INVENTION

The present invention relates to a holder for a surgically implantable device and particularly a holder for surgical screws.

### BACKGROUND OF THE INVENTION

It is common surgical practice to implant various prosthetic devices into a patient. Clearly, it is imperative that the prosthetic device implanted is sterile so as to prevent against infection.

In the field of orthopaedics, screws, nails, pins and other securing members are widely used, alone or in combination with adhesives, cements, or the like, for fastening one portion of bone to another or fixing a ligament. In addition to being sterile, such devices are preferably readily accessible to a surgeon and easily mounted on a driver for implantation into the body of the patient.

Conventionally, a tray containing the instruments required for the operation is prepared wherein the tray has a series of holes in its structure (up to a few hundred holes) which receive a screw or other like fastening device. The entire tray is sterilised and presented to the surgeon during the operation. At this time, the surgeon selects the screws required for the particular surgery, which in many cases may be as few as 10 out of the hundreds of screws. When the surgical procedure is finished, the tray is replenished with replacement screws and the tray subsequently re-sterilised for the next procedure. Accordingly, many of the screws on the tray are subjected to numerous rounds of sterilisation, which may result in damage to the screw such as wear on the thread and drive socket. Further, if a surgeon picks up the incorrect screw, he may replace it in the tray thereby contaminating the screw further. This is important when it is understood that specific objects are subjected to specific levels of sterilisation depending upon their level of contamination. Accordingly, where handling of the object has occurred, a different level of sterilisation. may be required. Furthermore, there is no way of distinguishing newly added screws from the “re-cycled” screws.

The present invention provides a holder for implantable surgical devices, which addresses the problems of the prior art.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY OF THE INVENTION

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In one aspect, the present invention consists in a holder for a surgically implantable device, said holder comprising a housing having a top portion and at least one side wall depending away from said top portion, said housing further including at least one internal receptacle member having an elongate hollow body defining at a proximal end an opening in the top portion, the elongate hollow body extending to a distal end spaced from the top portion.

In one embodiment, the holder further comprises a cover member which extends at least over the opening in the top portion such that a surgically implantable device received in the at least one receptacle member is retained therein by the cover member.

Preferably, the holder comprises a plurality of receptacle members. In a particularly preferred embodiment, the holder has four receptacle members.

The plurality of receptacle members may be arranged in a single row or a number of rows internal the housing. However, the invention is not to be limited to a particular arrangement of the plurality of receptacle members and any arrangement or combination of arrangements is envisaged.

The at least one receptacle member is preferably substantially cylindrical in shape with the proximal end having an area of greater diameter than the distal end. In another embodiment, the at least one receptacle member can be substantially frustoconical.

The provision of an area of greater diameter is particularly advantageous in an embodiment wherein the at least one receptacle member receives a surgical screw having a head and an elongate body tapering therefrom. The head of the screw has a larger diameter than the tapering body and therefore sits within a region of the proximal end of the at least one receptacle member. This has the added advantage of holding the head in a position so as to allow easy fixation to a screwdriver, while still protecting the underside of the head from accidental damage and preventing the threads from being coincidentally forced against the side walls of the screw holder. In a preferred embodiment, the portion of the holder cradling the screw head will be of the same dimensions as the underside of the screw head itself.

The implantable surgical device received in the at least one receptacle member may be a surgical screw, nail, pin and other securing members or any other device used in a surgical procedure.

Preferably, the at least one receptacle member is of a suitable length and/or dimension to enable a portion of the surgically implantable device received therein to sit flush with the top portion of the housing. In one embodiment, wherein the implantable device is a surgical screw, the head of the screw sits flush with the top portion of the housing.

Preferably, the at least one receptacle member is of a length that is shorter than the height of the at least one sidewall although it is equally envisaged that the at least one receptacle may extend beyond the distal edge of the sidewall or may extend for a length about equal or equal to the height of the at least one sidewall.

The cover member of the holder is preferably hingedly connected to the top portion of the housing and preferably hingedly connected to an outer edge of the top portion. Alternatively, the top portion may be hingedly connected to the at least one sidewall.

Typically, the cover member extends over the entire top portion although it is also envisaged that the cover member only extends over a region of the top portion which comprises the opening defined by the proximal end of the at least one receptacle member.

Preferably, the top portion includes a latch that receives a portion of the cover member such that the cover member is secured to the top portion and only released upon the exertion of a reasonable amount of pressure than would be expected during normal handling of the holder. Alternatively, the cover member can be arranged so as to comprise a spring element which both holds the cover member shut when closed or open when tilted up in a two phase manner. Accordingly, the cover member preferably does not release during normal handling of the holder or, for example, during sterilisation of the holder. This means that any implantable device such as a surgical screw which is received in the at least one receptacle member of the holder is prevented from falling out of the receptacle member during normal use.

The cover member may be secured to the top portion by any arrangement and is not limited by a particular mechanism.

The housing is preferably made from a plastics material such that it is relatively light weight and easy to handle. Other materials are envisaged although it is desirable that the material of the holder is softer than the material of the implantable device to be received by the at least one receptacle of the holder. In another embodiment, the plastic material may be colour coded, so as to further aid in the identification of the device type contained therein. Furthermore, this colour coding may be arranged such that it matches the colour of the driver tip used and the colour of the bone plates used in conjunction with any given screw from the coloured holder.

In a particularly preferred embodiment, the holder is made from a material which may be subjected to the high temperatures, irradiation, or chemicals required for initial sterilisation and it is further desirable that the holder is re-sterilisable by autoclave following use. In this regard, it is envisaged that only a portion of the implantable devices held in the holder may be used in any one procedure. If this is the case, the holder together with the remaining devices may be re-sterilised and used in a subsequent procedure. Furthermore, as there is no requirement to transfer the implantable devices to a separate holder for sterilisation, the risk of further contamination to the implantable devices from handling of said devices or damage to the structure of the devices (eg damage to a screw thread or head) is reduced.

Further, in one embodiment wherein the implantable devices are steam sterilised while retained within the holder, there may be less chance of foreign matter settling on the implantable devices as they are housed within the receptacle members of the holder.

In one embodiment of the invention, the at least one receptacle member may include a series of holes along the length of the elongate body. This embodiment has the advantage of facilitating steam or chemical penetration to the implantable device during sterilisation.

It is desirable that the holder of the present invention includes some form of identifier of the implantable device held therein. This is important to monitor details of the device, for example in the case of a screw, the screw diameter, length. Further, the identifier preferably includes details of the batch or lot number to aid in product recall should it be required. The details may be included on any part of the holder but preferably an external surface of the holder such as an external surface of the at least one sidewall or the top portion. The details may be stuck onto the holder or laser etched into the material of the holder. As mentioned above, because the entire holder may be re-sterilised, the identity of the implantable devices held therein may be maintained during the entire cycle of use and sterilisation.

Furthermore, in its original sterile packaging, the screw container may be contained within double layered sterile packaging, the outermost layer of which contains peel off labelling to facilitate the recording of the screw details in the medical records during the surgery or procedure.

### BRIEF DESCRIPTION OF THE DRAWINGS

By way of example only, a preferred embodiment of the invention is now described with reference to the accompanying drawings, in which:

FIG. 1 is a perspective view of one embodiment of the holder of the present invention with the cover in a closed position;

FIG. 2 is a top plan view of the holder of FIG. 1;

FIG. 3 is a side view of the holder of FIG. 1;

FIG. 4 is an inverse plan view of the holder of FIG. 1; and

FIG. 5 is a perspective view of the holder of FIG. 1 depicting the cover in an open position.

### PREFERRED MODE OF CARRYING OUT THE INVENTION

One embodiment of a holder for a surgically implantable device of the present invention is generally depicted as 10 in the accompanying drawings.

The holder comprises a housing 11 having a top portion 12 and side walls 13 depending from the top portion 12. The housing 11 further includes a series of internal receptacle members 14 each having an elongate hollow body defining at a proximal end 16 an opening 17 in the top portion 12. The elongate hollow body of each receptacle member 14 extends to a distal end 18 which is spaced from the top portion 12.

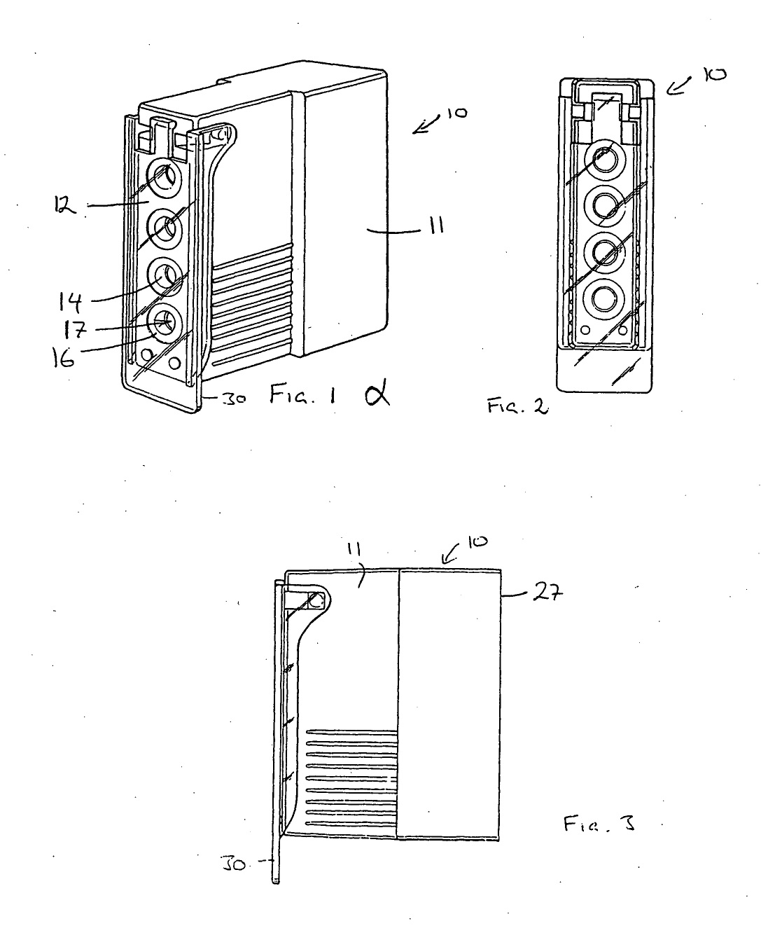

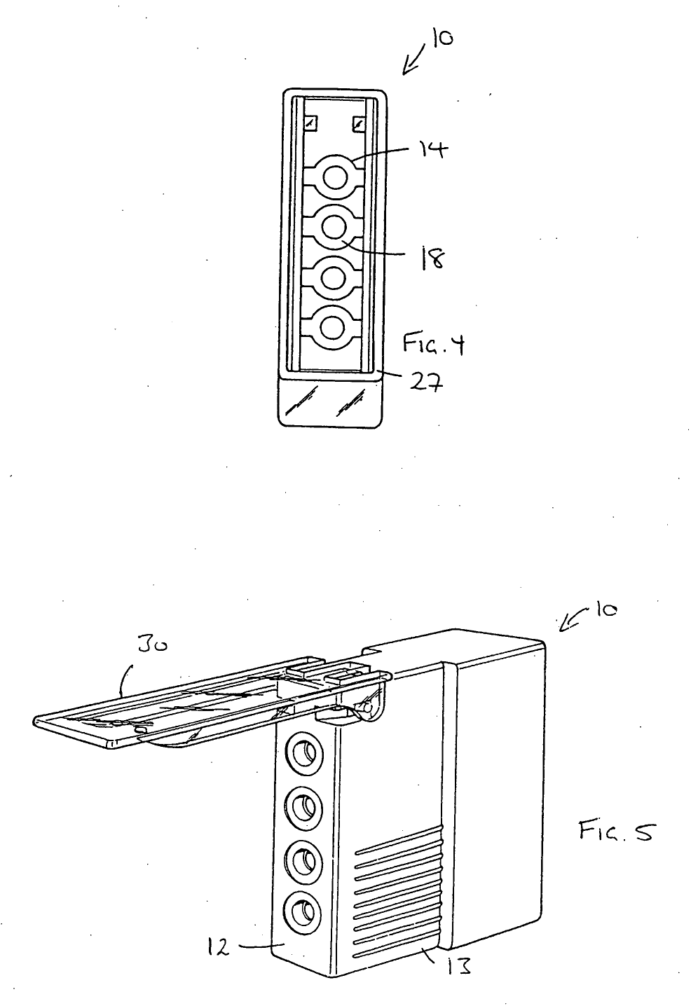

The holder 10 further comprises a transparent cover member 30 which extends over at least the openings 17 such that surgically implantable devices received in the receptacle members 14 are retained therein by the cover member. As depicted, the cover member 30 is hingedly connected to the housing 11 and can be moved from a closed position (depicted in FIG. 1) to an open position (depicted in FIG. 5).

The holder 10 depicted in the drawings has four receptacle members 14.

In the depicted embodiment, each receptacle member 14 is substantially cylindrical in shape with the proximal end 16 having a slightly greater diameter than the distal end 18. This is particularly advantageous in the depicted embodiment wherein the each receptacle member receives a surgical screw having a head and an elongate body tapering therefrom. The head of the screw typically has a larger diameter than the tapering body and therefore sits within a region of the proximal end 16 of each receptacle member 14.

It can also be seen that each receptacle member 14 is of a suitable length and/or dimension to enable the head of the screw to sit flush with the top portion 12 of the housing 11.

In the depicted embodiment, each receptacle member 14 extends downwardly from the top portion 12 for a length that is shorter than the length of the sidewall 13 measured from the top portion 12 to the distal edge 27.

The housing 11 is preferably made from a plastics material such that it is relatively light weight and easy to handle. Other materials are envisaged although it is desirable that the material of the holder is softer than the material of the implantable device to be received by the at least one receptacle of the holder.

Use of the device results in there being no requirement to transfer the implantable devices to a separate holder for sterilisation. This reduces the risk of further contamination to the implantable devices from handling of said devices or damage to the structure of the devices (eg damage to a screw thread or head).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A holder for a surgically implantable device, said holder comprising a housing having a top portion and at least one side wall depending away from said top portion, said housing further including at least one internal receptacle member having an elongate hollow body defining at a proximal end an opening in the top portion, the elongate hollow body extending to a distal end spaced from the top portion.

2. The holder of claim 1 wherein the holder further comprises a cover member which extends at least over said at least one opening in the top portion such that a surgically implantable device received in the at least one receptacle member is retained therein by the cover member.

3. The holder of claim 1 wherein the holder further comprises a plurality of receptacle members.

4. The holder of claim 3 wherein the holder has four receptacle members.

5. The holder of claim 3 wherein the plurality of receptacle members are arranged in a single row internal the housing.

6. The holder of claim 1 wherein the at least one receptacle member is preferably substantially cylindrical in shape with the proximal end having an area of greater diameter than the distal end.

7. The holder of claim 1 wherein the implantable surgical device received in the at least one receptacle member is selected from the group comprising a surgical screw, nail, pin, and any other device used in a surgical procedure.

8. The holder of claim 1 wherein the at least one receptacle member is of a suitable length and/or dimension to enable a portion of the surgically implantable device received therein to sit flush with the top portion of the housing.

9. The holder of claim 1 wherein the at least one receptacle member is of a length that is shorter than the height of the at least one sidewall.

10. The holder of claim 2 wherein the cover member of the holder is hingedly connected to the housing.

11. The holder of claim 10 wherein the cover member extends over the entire top portion of the holder.

12. The holder of claim 10 wherein the cover member further comprises a spring element which both holds the cover member shut when closed or open when tilted up in a two phase manner.

13. The holder of claim 1 wherein the holder is made of a plastic material that is colour coded to aid in the identification of the device type contained therein.

14. The holder of claim 1 wherein the at least one receptacle member has a series of holes along the length of the elongate body that facilitate steam or chemical penetration to the implantable device during sterilisation.

15. The holder of claim 1 wherein the holder has an identifier for the implantable device held therein.
